(19) United States
(12) Patent Application Publication
(10) Pub. No.: US 2023/0346803 A1
(43) Pub. Date: Nov. 2, 2023

(54) COMPOSITIONS OF BERBERINE URSODEOXYCHOLATE AND METHODS THEREOF FOR TREATING PRIMARY SCLEROSING CHOLANGITIS

(71) Applicant: SHENZHEN HIGHTIDE BIOPHARMACEUTICAL LTD., Shenzhen (CN)

(72) Inventor: Liping Liu, Shenzhen (CN)

(21) Appl. No.: 18/202,033

(22) Filed: May 25, 2023

Related U.S. Application Data
(63) Continuation-in-part of application No. PCT/CN2022/074670, filed on Jan. 28, 2022.
(60) Provisional application No. 63/347,245, filed on May 31, 2022.
(60) Provisional application No. 63/142,750, filed on Jan. 28, 2021.

(51) Int. Cl. A61K 31/585; A61P 1/16

(52) U.S. Cl. CPC A61K 31/585; A61P 1/16

(57) ABSTRACT

The invention provides pharmaceutical compositions of berberine ursodeoxycholate (BUDCA) and methods of use thereof for the treatment primary sclerosing cholangitis, and related diseases and conditions.

### PRIORITY CLAIMS AND RELATED PATENT APPLICATIONS

This application claims the benefit of priority to U.S. Provisional Application Serial No. 63/347,245, filed May 31, 2022 and claims the benefit of priority to PCT Int′l Application No. PCT/CN2022/074670, filed Jan. 28, 2022, which claims priority to U.S. Provisional Application Serial No. 63/142,750, filed Jan. 28, 2021, the entire content of each of which is incorporated herein by reference.

### TECHNICAL FIELD OF THE INVENTION

The invention generally relates to compositions of berberine ursodeoxycholate (BUDCA), and methods for therapeutic uses thereof. In particular, the invention relates to pharmaceutical compositions and methods of use of BUDCA for the treatment primary sclerosing cholangitis (PSC), and related diseases and conditions, as monotherapy, in combination therapy with other agents, or an adjuvant.

### BACKGROUND OF THE INVENTION

PSC is a progressive inflammatory and cholestatic liver disease that damages the bile ducts due to inflammation, which causes scars within the bile ducts leading to serious liver damage. This cholestatic disease leads to fibrosis and ultimately liver failure, cirrhosis, and an increased risk of malignancy. Liver failure often occur ten to fifteen years after diagnosis. Patients with PSC continue to have an increased risk of, and many do suffer from, a number of serious complications during the progression of the disease. For example, PSC may lead to bile duct cancer. (Primary sclerosing cholangitis. Lancet. 2018;391(10139):2547-2559.; AASLD practice guidelines: Diagnosis and management of primary sclerosing cholangitis. Hepatology. 2010;51(2):660-78.; The challenges in primary sclerosing cholangitis--aetiopathogenesis, autoimmunity, management and malignancy. Journal of Hepatology 48 Suppl 1(1):S38-57.) Although the pathogenesis of PSC is not entirely clear, it does involve gut dysbiosis, alterations in bile acid metabolism and immune-mediated bile duct injury.

Despite continued efforts over the past decades in understanding and management of PSC, currently there are no FDA-approved therapeutic agents that effectively treat PSC. There are no specific treatments available that stop or slow the progression of the disease. Many people with PSC will ultimately need a liver transplant.

There is a significant unmet and urgent need for novel therapeutics and methods that can be used effectively to treat and manage PSC, or related diseases and conditions.

### SUMMARY OF THE INVENTION

The invention is based in part on novel compositions and methods of use of BUDCA for treating PSC, as a single therapeutic agent or optionally in combination with or as adjunctive to other agents.

In one aspect, the invention generally relates to a method for treating PSC. The method comprises administering to a subject in need thereof a therapeutically effective amount of a pharmaceutical composition comprising BUDCA.

In another aspect, the invention generally relates to a method for reducing serum alkaline phosphatase (ALP) in a subject suffering from PSC, comprising administering to a subject in need thereof a therapeutically effective amount of a pharmaceutical composition comprising BUDCA.

In yet another aspect, the invention generally relates to a method for reducing cholestasis and/or hepatocellular inflammation in patients with PSC and elevated ALP, comprising administering to a subject in need thereof a therapeutically effective amount of a pharmaceutical composition comprising BUDCA.

In yet another aspect, the invention generally relates to a method for reducing AST, ALT and/or ALP in patients with PSC and elevated ALP, comprising administering to a subject in need thereof a therapeutically effective amount of a pharmaceutical composition comprising BUDCA.

In yet another aspect, the invention generally relates to a unit dosage form suitable for administration in treatment of PSC.

In yet another aspect, the invention generally relates to a unit dosage form suitable for reducing ALP in a subject suffering from PSC.

In yet another aspect, the invention generally relates to a unit dosage form suitable for reducing cholestasis and/or hepatocellular inflammation in patients with PSC and elevated ALP.

In yet another aspect, the invention generally relates to a unit dosage form suitable for reducing AST, ALT and/or ALP in patients with PSC and elevated ALP.

In yet another aspect, the invention generally relates to a unit dosage form suitable for administration in treatment of PSC.

In yet another aspect, the invention generally relates to a unit dosage form suitable for reducing ALP in a subject suffering from PSC.

In yet another aspect, the invention generally relates to a unit dosage form suitable for reducing cholestasis and/or hepatocellular inflammation in patients with PSC and elevated ALP.

In yet another aspect, the invention generally relates to a unit dosage form suitable for reducing AST, ALT and/or ALP in patients with PSC and elevated ALP.

### BRIEF DESCRIPTION OF THE DRAWINGS

FIG. 1 shows the subject disposition in the clinical trial disclosed in the Examples section.

FIG. 2 shows certain exemplary results on ALP response.

FIG. 3 shows certain exemplary results on ALP response.

FIG. 4 shows certain exemplary results on GGT response.

FIG. 5 shows certain exemplary results on ALP response.

FIG. 6 shows certain exemplary results on GGT response.

FIG. 7 shows certain exemplary results on liver-related enzymes showing improvement at Week 6.

FIG. 8 shows certain exemplary results on ALP response during the Treatment Extension (Period 2) and Randomized Withdrawal (Period 3) Phases.

FIG. 9 shows certain exemplary results on ALP response.

FIG. 10 shows certain exemplary results on ALP response.

FIG. 11 depicts a unit cell of single crystalline Form A of BUDCA.

### DEFINITIONS

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Ranges provided herein are understood to be shorthand for all of the values within the range. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, “at least” a specific value is understood to be that value and all values greater than that value.

As used herein, “more than one” is understood as 2, 3, 4, 5, 6, 7, 8, 9, 10, 11, 12, 13, 14, 15, 16, 17, 18, 19, 20, 25, 30, 40, 50, 100, etc., or any value therebetween.

In this specification and the appended claims, the singular forms “a,” “an,” and “the” include plural reference, unless the context clearly dictates otherwise.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Unless specifically stated or obvious from context, as used herein, the term “or” is understood to be inclusive.

Any compositions or methods disclosed herein can be combined with one or more of any of the other compositions and methods provided herein.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer’s solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “therapeutically effective amount” refers to the dose of a therapeutic agent or agents sufficient to achieve the intended therapeutic effect with minimal or no undesirable side effects. A therapeutically effective amount can be readily determined by a skilled physician, e.g., by first administering a low dose of the pharmacological agent(s) and then incrementally increasing the dose until the desired therapeutic effect is achieved with minimal or no undesirable side effects.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. The treatment can be any reduction and can be, but is not limited to, the complete ablation of the disease or the symptoms of the disease. Treating or treatment thus refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient’s physical or mental well-being. The treatment or amelioration of symptoms can be based on objective or subjective parameters, for example, the results of a physical examination, neuropsychiatric exams, and/or a psychiatric evaluation. As compared with an equivalent untreated control, such reduction or degree of amelioration may be at least 5%, 10%, 20%, 40%, 50%, 60%, 80%, 90%, 95%, or 100% as measured by any standard technique.

Treatment methods include administering to a subject a therapeutically effective amount of a compound described herein. The administering step may be a single administration or may include a series of administrations. The length of the treatment period depends on a variety of factors, such as the severity of the condition, the patient’s age, the concentration of the compound, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required. For example, the compositions are administered to the subject in an amount and for a duration sufficient to treat the patient.

As used herein, the terms “isolated” or “purified” refer to a material that is substantially or essentially free from components that normally accompany it in its native state. Purity and homogeneity are typically determined using analytical chemistry techniques such as polyacrylamide gel electrophoresis or high-performance liquid chromatography.

As used herein, the term “subject” refers to any animal (e.g., a mammal), including, but not limited to humans, non-human primates, rodents, and the like, which is to be the recipient of a particular treatment. A subject to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or other non-human animals, for example, non-human mammals (e.g., primates (e.g., cynomolgus monkeys, rhesus monkeys); commercially relevant mammals such as cattle, pigs, horses, sheep, goats, cats, and/or dogs), rodents (e.g., rats and/or mice), etc. In certain embodiments, the non-human animal is a mammal. The non-human animal may be a male or female at any stage of development. A non-human animal may be a transgenic animal. Typically, the terms “subject” and “patient” are used interchangeably herein in reference to a human subject.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The present invention is based in part on the unexpected discovery of a novel treatment for PSC, as evidenced by data obtained from phase 2 randomized, placebo-controlled clinical trials where BUDCA was orally administered and well tolerated. The clinical trial results demonstrated that BUDCA was effective in treating PSC. For example, clinical studies showed significant reductions in ALP at two dose regimens of BUDCA. In addition, dose-dependent improvements were clearly observed in serum markers of cholestasis and liver injury, as measured by aspartate aminotransferase (AST), alanine aminotransferase (ALT), and gamma-glutamyl transpeptidase (GGT). Thus, the present invention provides a novel and ground-breaking therapeutic agent, and compositions and methods thereof, for treating PSC.

Elevations of liver-derived ALP are the biochemical hallmark of cholestasis and an elevation in serum ALP is one of the first externally detectable signs of PSC. In the presence of cholestatic liver injury the damaged liver regurgitates hepatic ALP back into serum. (Induction of rat liver alkaline phosphatase: the mechanism of the serum elevation in bile duct obstruction. J Clin Invest. 1970;49(3):508-16.) The elevation in serum ALP in hepatobiliary disease also results from increased de novo synthesis in the liver followed by release into the circulation. (Serum alkaline phosphatase--another piece is added to the puzzle, Hepatology 1986 May-Jun;6(3):526-8.) The degree and persistence of the ALP elevation is correlated with the progression of liver and biliary disease. ALP has commonly been used as an endpoint in interventional studies in PSC because of its role as the biochemical hallmark of cholestasis. (Surrogate endpoints for clinical trials in primary sclerosing cholangitis: Review and results from an International PSC Study Group consensus process, Hepatology 2016;63(4):1357-67; Design and endpoints for clinical trials in primary sclerosing cholangitis, Hepatology 2018;68(3):1174-88.; European Medicines Agency (EMA); Reflection paper on regulatory requirements for the development of medicinal products for chronic non-infectious liver diseases (PBC, PSC, NASH). November 2018.)

BUDCA is a new molecular entity and can be administered orally. The compound was disclosed in WO 2016/015634 A1 (PCT/CN2015/085350) and WO 2018/205987 A1 (PCT/CN2018/086461), the content of each of which is incorporated herein by reference in its entirety. BUDCA is an ionic salt of berberine (BBR) and ursodeoxycholic acid (UDCA), represented by:

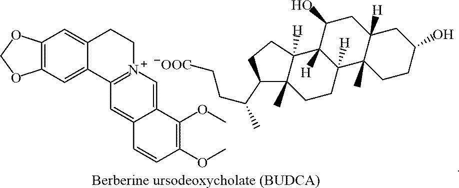

In one aspect, the invention generally relates to a method for treating PSC. The method comprises administering to a subject in need thereof a therapeutically effective amount of a pharmaceutical composition comprising BUDCA.

In another aspect, the invention generally relates to a method for reducing ALP in a subject suffering from PSC, comprising administering to a subject in need thereof a therapeutically effective amount of a pharmaceutical composition comprising BUDCA.

In yet another aspect, the invention generally relates to a method for reducing cholestasis and/or hepatocellular inflammation in patients with PSC and elevated ALP, comprising administering to a subject in need thereof a therapeutically effective amount of a pharmaceutical composition comprising BUDCA.

In yet another aspect, the invention generally relates to a method for reducing AST, ALT and/or ALP in patients with PSC and elevated ALP, comprising administering to a subject in need thereof a therapeutically effective amount of a pharmaceutical composition comprising BUDCA.

In yet another aspect, the invention generally relates to a method for reducing liver injury in patients with PSC, comprising administering to a subject in need thereof a therapeutically effective amount of a pharmaceutical composition comprising BUDCA.

In certain embodiments, the subject has not been previously treated for PSC.

In certain embodiments, the subject has not been previously treated with UDCA.

In certain embodiments, the subject has been previously treated with UDCA.

In certain embodiments, the subject has been previously treated with UDCA, and the subject should be treated with BUDCA for at least 12 weeks.

In certain embodiments, the subject has been previously treated with UDCA, and the subject should be treated with BUDCA for at least 18 weeks.

In certain embodiments, the subject suffers from an elevated ALP of above 3-fold ULN at baseline.

In certain embodiments for treating PSC and/or reducing ALP in a subject suffering from PSC, the subject is administered from about 250 mg/day to about 5,000 mg/day (e.g., about 250 mg/day to about 4,000 mg/day, about 500 mg/day to about 3,000 mg/day, about 500 mg/day to about 2,500 mg/day, about 500 mg/day to about 2,000 mg/day, about 500 mg/day to about 1,500 mg/day, about 250 mg/day to about 500 mg/day, about 500 mg/day to about 1,000 mg/day, about 1,000 mg/day to about 2,000 mg/day, about 2,000 mg/day to about 3,000 mg/day, about 3,000 mg/day to about 5,000 mg/day) of BUDCA.

In certain embodiments, BUDCA may be administered to the subject once a day (QD), twice a day (BID), three times a day (TID) or four times a day (QID), or as otherwise as deemed appropriated by a qualified healthcare professional.

In certain embodiments for treating PSC and/or reducing ALP in a subject suffering from PSC, BUDCA is in the form of Form A.

In certain embodiments, BUDCA is a hydrate.

In certain embodiments, BUDCA is a hemi-nonahydrate.

In yet another aspect, the invention generally relates to a unit dosage form suitable for administration in treatment of PSC.

In yet another aspect, the invention generally relates to a unit dosage form suitable for reducing ALP in a subject suffering from PSC.

In yet another aspect, the invention generally relates to a unit dosage form suitable for reducing cholestasis and/or hepatocellular inflammation in patients with PSC and elevated ALP.

In yet another aspect, the invention generally relates to a unit dosage form suitable for reducing AST, ALT and/or ALP in patients with PSC and elevated ALP.

In certain embodiments, the unit dosage form is in the form of a capsule.

In certain embodiments, the unit dosage form is in the form of a tablet.

In certain embodiments, the unit dosage form comprises BUDCA and one or more pharmaceutically acceptable excipients, carriers, or diluents.

In certain embodiments, the subject additionally suffers from hyperlipidemia and/or diabetes.

In yet another aspect, the invention generally relates to use of BUDCA for treating PSC and/or for reducing ALP in a subject suffering from PSC.

In yet another aspect, the invention generally relates to use of BUDCA for reducing ALP in a subject suffering from PSC.

In yet another aspect, the invention generally relates to use of BUDCA for reducing cholestasis and/or hepatocellular inflammation in patients with PSC and elevated ALP.

In yet another aspect, the invention generally relates to use of BUDCA for reducing AST, ALT and/or ALP in patients with PSC and elevated ALP.

Various solid and crystalline forms of BUDCA may be employed in the invention disclosed herein.

In certain embodiments, Form A of BUDCA, having an X-ray powder diffraction (XRPD) pattern comprising one or more peaks at 2θ values selected from the group consisting of: 3.98, 7.06, 7.34, 7.93, 8.79, 9.47, 11.70, 11.94, 12.34, 12.55, 13.90, 14.17, 15.14, 15.50, 16.16, 16.54, 16.78, 17.53, 17.67, 18.23, 19.03, 19.98, 20.87, 21.13, 21.96, 23.49, 24.24, 24.97, 25.50, 26.63, 27.60, 28.06, 28.63, 29.40 and 30.49 ° (± 0.2°) obtained using Cu Kα radiation (λ₁ = 1.540598 Å, λ₂ = 1.544426 Å, intensity ratio λ₂/λ₁ = 0.50), is employed.

In certain embodiments, Form B of BUDCA, having an X-ray powder diffraction (XRPD) pattern comprising one or more peaks at 2θ values selected from the group consisting of: 7.39, 9.31, 12.41, 13.14, 14.37, 14.76, 15.53, 18.65, 21.79, 22.87, 25.27, 25.53 and 28.12 ° (± 0.2°) obtained using Cu Kα radiation (λ₁ = 1.540598 Å, λ₂ = 1.544426 Å, intensity ratio λ₂/λ₁ = 0.50), is employed.

In certain embodiments, Form C of BUDCA, having an X-ray powder diffraction (XRPD) pattern comprising one or more peaks at 2θ values selected from the group consisting of: 7.23, 10.42, 12.10, 13.37, 14.24, 14.48, 15.28, 15.95, 17.00, 18.17, 20.12, 21.77 and 25.47 ° (± 0.2°) obtained using Cu Kα radiation (λ₁ = 1.540598 Å, λ₂ = 1.544426 Å, intensity ratio λ₂/λ₁ = 0.50), is employed.

In certain embodiments, Form D of BUDCA, having an X-ray powder diffraction (XRPD) pattern comprising one or more peaks at 2θ values selected from the group consisting of: 4.24, 6.79, 8.50, 10.25, 11.50, 13.62, 14.74, 15.20, 17.92, 18.39, 22.91 and 25.73 ° (± 0.2°) obtained using Cu Kα radiation (λ₁= 1.540598 Å, λ₂ = 1.544426 Å, intensity ratio λ₂/λ₁ = 0.50), is employed.

In certain embodiments, Form E of BUDCA, having an X-ray powder diffraction (XRPD) pattern comprising one or more peaks at 2θ values selected from the group consisting of: 8.59, 10.55, 11.36, 11.86, 12.46, 13.08, 13.38, 14.34, 15.57, 17.24, 17.72, 18.43, 19.66, 19.84, 20.35, 20.91, 21.36, 21.95, 23.21, 24.67, 25.04, 25.82, 26.12, 27.01, 27.84, 28.97, 30.35, 33.33, 34.54 and 36.06 ° (± 0.2°) obtained using Cu Kα radiation (λ₁= 1.540598 Å, λ₂ = 1.544426 Å, intensity ratio λ₂/λ₁ = 0.50), is employed.

In certain embodiments, Form H of BUDCA, having an X-ray powder diffraction (XRPD) pattern comprising one or more peaks at 2θ values selected from the group consisting of: 13.05, 14.63 and 25.46 ° (± 0.2°) obtained using Cu Kα radiation (λ₁ = 1.540598 Å, λ₂ = 1.544426 Å, intensity ratio λ₂/λ₁ = 0.50), is employed.

In certain embodiments, Form I of BUDCA, having an X-ray powder diffraction (XRPD) pattern comprising one or more peaks at 2θ values selected from the group consisting of: 4.19, 7.64, 10.03, 13.32, 13.84, 14.83, 16.73, 22.73, 25.61 and 28.57 ° (± 0.2°) obtained using Cu Kα radiation (λ₁= 1.540598 Å, λ₂ = 1.544426 Å, intensity ratio λ₂/λ₁ = 0.50), is employed.

In certain embodiments, Form J of BUDCA, having an X-ray powder diffraction (XRPD) pattern comprising one or more peaks at 2θ values selected from the group consisting of: 4.61, 6.32, 7.38, 8.22, 9.21, 10.57, 11.73, 12.13, 12.62, 12.96, 13.87, 14.55, 14.78, 15.81, 16.48, 17.69, 18.39, 19.01, 20.06, 21.25, 22.13, 23.20, 24.47, 24.89, 26.31, 27.98, 30.25 and 33.35 ° (± 0.2°) obtained using Cu Kα radiation (λ₁= 1.540598 Å, λ₂ = 1.544426 Å, intensity ratio λ₂/λ₁ = 0.50), is employed.

In certain embodiments, Form P of BUDCA, having an X-ray powder diffraction (XRPD) pattern comprising one or more peaks at 2θ values selected from the group consisting of: 3.11, 5.01, 5.78, 7.26, 9.20, 10.10, 10.79, 11.65, 13.70, 14.59, 15.22, 16.19, 16.54, 17.05, 18.06, 18.68, 20.52, 21.09, 21.73, 22.49, 24.73, 25.42, 25.94 and 30.11 ° (± 0.2°) obtained using Cu Kα radiation (λ₁= 1.540598 Å, λ₂ = 1.544426 Å, intensity ratio λ₂/λ₁ = 0.50), is employed.

In certain embodiments, Form W of BUDCA, having an X-ray powder diffraction (XRPD) pattern comprising one or more peaks at 2θ values selected from the group consisting of: 6.49, 7.16, 8.51, 10.21, 12.01, 13.13, 13.90, 14.42, 15.18, 15.57, 16.03, 16.45, 16.74, 17.08, 17.85, 18.39, 19.61, 20.43, 21.39, 21.70, 23.51 and 25.21° ( ± 0.2°) obtained using Cu Kα radiation (λ₁= 1.540598 Å, λ₂ = 1.544426 Å, intensity ratio λ₂/λ₁ = 0.50), is employed.

In certain embodiments, Form X of BUDCA, having an X-ray powder diffraction (XRPD) pattern comprising one or more peaks at 2θ values selected from the group consisting of: 3.63, 6.61, 7.24, 10.49, 11.95, 13.51, 14.26, 14.54, 15.14, 16.01, 16.82, 18.28, 20.26, 21.08, 21.49, 21.90, 25.60, 26.40, 27.31, 29.34, 30.59, 31.01, 34.04, 34.68 and 36.91° (± 0.2°) obtained using Cu Kα radiation (λ₁= 1.540598 Å, λ₂ = 1.544426 Å, intensity ratio λ₂/λ₁ = 0.50), is employed.

In certain embodiments, the solid or crystalline form (e.g., Form A of BUDCA) is a hydrate of BUDCA. In certain embodiments, the solid or crystalline form (e.g., Form A of BUDCA) is a hemi-nonahydrate of BUDCA.

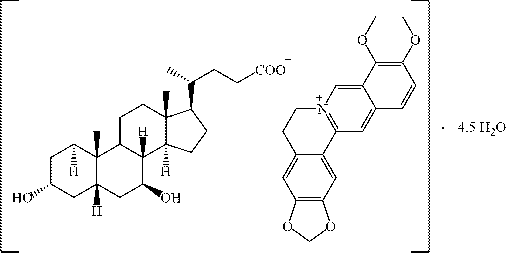

### Hemi-Nonahydrate of BUDCA

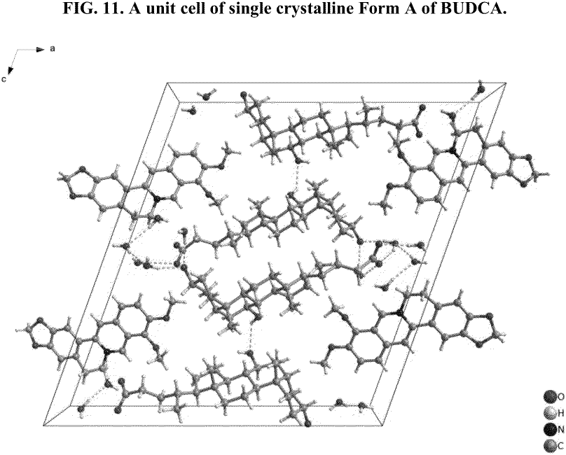

In certain embodiments, the solid or crystalline form (e.g., Form A of BUDCA) is crystalline. In certain embodiments, the crystalline form is characterized in a monoclinic crystal system and P2₁ space group. In certain embodiments of the crystalline form, each unit cell contains two asymmetric units and there are two BBR cations, two UDCA anions and nine H₂O molecules per asymmetric unit, and four BBR cations, four UDCA anions and eighteen H₂O molecules per unit cell. (FIG. 11.)

Possible formulations include those suitable for oral, sublingual, buccal, parenteral (for example subcutaneous, intramuscular, or intravenous), rectal, topical including transdermal, intranasal and inhalation administration. Most suitable means of administration for a particular patient will depend on the nature and severity of the disease or condition being treated or the nature of the therapy being used and on the nature of the active compound.

The following examples are meant to be illustrative of the practice of the invention and not limiting in any way.

### EXAMPLES

A Phase 2, multicenter, dose-ranging, 18-week study was conducted comparing two dose regimens of BUDCA (500 mg BID and 1,000 mg BID) to placebo in adult subjects with PSC.

### Materials

BUDCA drug product (also herein referred to as HTD1801) is a film-coated tablet containing 250 mg of berberine ursodeoxycholate.

Placebo: 250 mg BUDCA-matching tablets.

### Study Design

The clinical study design consisted of an initial randomized, double-blind, placebo-and dose-controlled, parallel-group period (Period 1), followed by a dose-controlled extension period (Period 2), which is followed by a placebo-controlled randomized withdrawal period (Period 3).
Period 1: Subjects were randomized to receive BID doses of 500 mg, 1,000 mg, or matching placebo for 6 weeks. The primary analysis was based on this initial 6-week randomized, double-blind, placebo- and dose-controlled, parallel-group period.
Period 2: Subjects previously randomized to 500 mg BID or 1,000 mg BID continued for 6 more weeks at that previous dose, while subjects previously randomized to placebo were re-randomized to receive 6 weeks of either 500 mg BID or 1,000 mg BID.
Period 3: Subjects were re-randomized to either continue on the active treatment they had received in Period 2 or be assigned to placebo.

Patients with a known diagnosis of PSC based upon magnetic resonance cholangiopancreatography (MRCP) or endoscopic retrograde cholangiopancreatography (ERCP) findings were eligible for inclusion in this study. To be eligible for randomization into the study, serum ALP levels at screening had to be elevated at least above 1.5 times the upper limit of normal (ULN). Patients were excluded if they had a known or suspected dominant biliary stricture, any other identifiable cause of liver disease, including autoimmune hepatitis, small-duct PSC, IgG4-related cholangitis or evidence of decompensated liver disease.

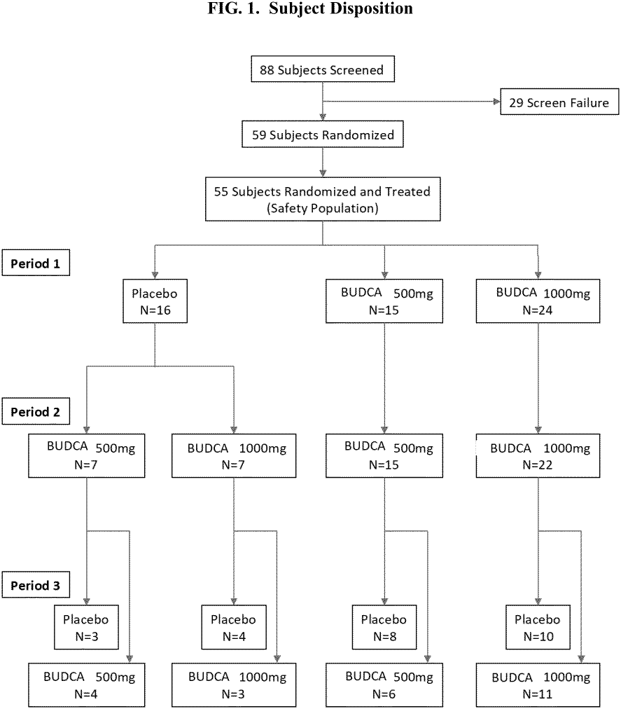

A summary of subject disposition is provided in FIG. 1. The primary endpoint was the absolute change in ALP from Baseline to Week 6 (Period 1).

For this study, 88 subjects with PSC were screened, 59 were randomized, 55 received at least 1 dose of study drug (safety population), and 54 had at least 1 post-dose efficacy assessment (modified intent-to-treat (mITT) population). The baseline characteristics of the study population are shown in Table 1.

At the time of enrollment, the mean serum ALP for the safety population was 375 U/L, ranging between 122 and 1048 U/L. Four patients (6.8%) had screening ALP>1.5xULN, but Day 0 ALP<1.5xULN (Placebo; n=2, BUDCA 1,000 mg BID, n=2).

TABLE 1

Baseline Characteristics
BUDCA

| | Placebo (n=16) | 500 mg (n=15) | 1,000 mg (n=24) |
|---|---|---|---|
| Mean Age (range, yrs) | 40 (21-72) | 43 (29-69) | 45 (24-75) |
| Mean ALP (U/L) at Baseline (range) | 414 (138-1048) | 397 (237-773) | 335 (122-882) |
| Mean GGT (U/L) at Baseline (range) | 497 (119-1563) | 742 (76-3535) | 554 (64-2015) |
| Mean ALT (U/L) at Baseline (range) | 93 (24-166) | 153 (35-420) | 112 (31-363) |
| Mean Bilirubin (mg/dL) at Baseline | 0.8 (0.4-1.4) | 1.1 (0.5-2.6) | 0.8 (0.3-1.4) |

All statistical tests were two-sided with an alpha level of 0.05. The statistical analyses were conducted with the SAS® System version 9.4 or higher. All analyses were subject to formal verification procedures.

### Results

This study met the primary efficacy endpoint, the absolute change in ALP from Baseline to Week 6 in the double-blind parallel-group period (Period 1): There was a clinically meaningful and statistically significant decrease in ALP from Baseline to Week 6 with both doses of BUDCA compared to an increase in ALP in the placebo group, and a clear signal for benefit in subjects who had not been on UDCA prior to enrollment in this study.

Greater ALP responses in the active treatment groups compared to placebo were apparent as early as Week 2 and sustained through Week 6, and more subjects receiving active treatment met the secondary ALP endpoints (See FIG. 3) at Week 6 than subjects receiving placebo. A dose response was also evident across multiple efficacy endpoints, with ~2-fold greater response rate with the 1,000 mg BID compared with the 500 mg BID dose for each endpoint at Week 6.

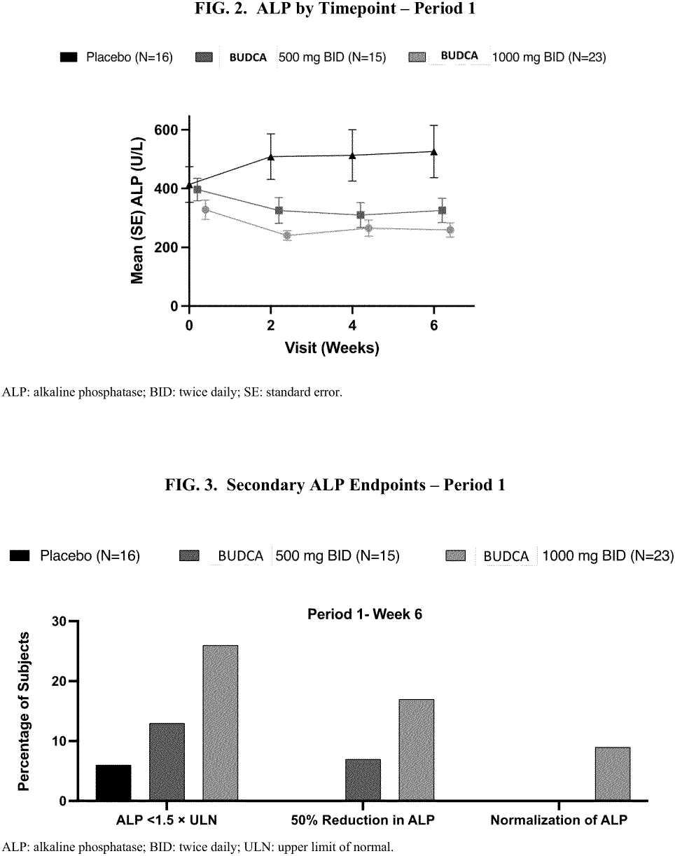

Exemplary results on the ALP response are shown in FIG. 2 and FIG. 3.

GGT, ALT and AST also were reduced with both doses of BUDCA compared to a worsening in liver biochemistries in subjects treated with placebo. With BUDCA treatment, reductions in GGT, ALT, and AST were apparent as early as Week 2 and sustained through Week 6 in Period 1.

Trends for GGT were similar to those observed for ALP. This was reflected both in terms of improved GGT in the active treatment arms and worsened GGT in the placebo arm. The GGT response appeared to be greater numerically with BUDCA 1,000 mg BID as compared to BUDCA 500 mg BID.

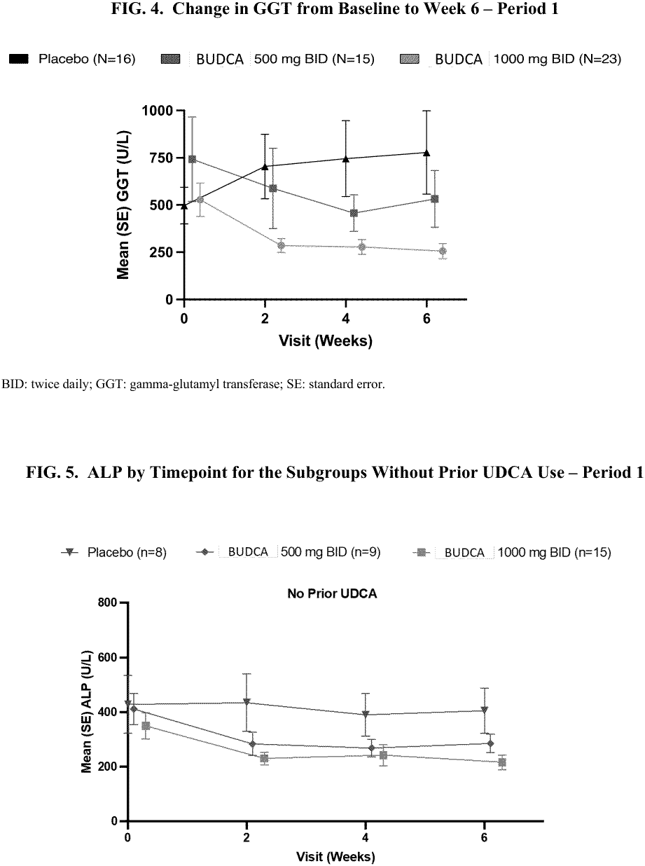

As with Baseline ALP, Baseline GGT was considerably lower in the BUDCA 1,000 mg BID group. Subjects receiving either BUDCA 500 mg BID or 1,000 mg BID had a greater absolute and percent decrease from baseline in GGT compared to placebo as early as Week 2 as well as at all other timepoints. At Week 6, percent change from Baseline was -21% for BUDCA 500 mg BID, -40% for BUDCA 1,000 mg BID, and 47% for placebo. Exemplary results on the GGT response are shown in FIG. 4.

Subjects who received active treatment without prior UDCA use showed clinically meaningful reductions in ALP as early as Week 2 and at every timepoint thereafter compared to subjects who received placebo. Over half of those assigned active treatment achieved a 25% reduction in ALP, compared to 0 subjects who received placebo. BUDCA 1,000 mg BID was more effective than BUDCA 500 mg BID for meeting the other secondary ALP responder endpoints in subjects without prior UDCA use. FIG. 5 shows exemplary results on the ALP response in subgroups of patients without prior UDCA use.

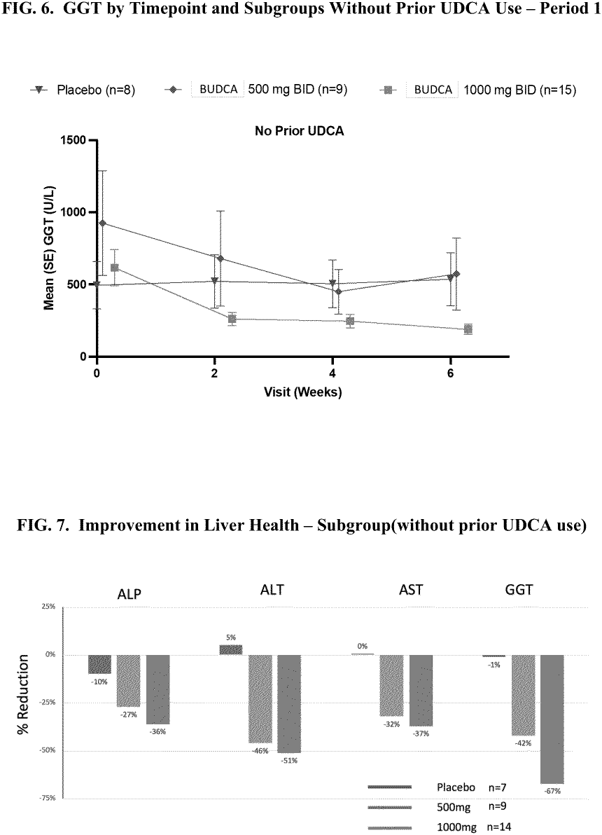

Trends in GGT response to BUDCA were similarly impacted by UDCA status prior to receiving active treatment. Subjects who received active treatment without prior UDCA use showed clinically meaningful reductions in GGT as early as Week 2 and at every timepoint thereafter compared to minimal change for subjects who received placebo. In these subjects, percent change in GGT at Week 6 was -42%, -67%, and 0% with BUDCA 500 mg BID, BUDCA 1,000 mg BID, and placebo, respectively. FIG. 6 shows exemplary results on the GGT response in subgroup of patients without prior UDCA use.

Other markers of liver injury (such as ALT, AST) were similarly improved with BUDCA in all enrolled patients and the subgroup of patients without prior UDCA use. The improvement of liver-related enzymes in the subgroup of patients without prior UDCA use is shown in FIG. 7.

There were 8/24 patients, 7/15 patients and 9/16 patients had ALP above 3x (equals to 3-fold) ULN in the HTD1801 1,000 mg BID, HTD1801 500 mg BID and placebo groups, respectively. A history of prior UDCA use was present in ⅛, 2/7 and 4/9 patients with ALP above 3x ULN at baseline and 7/16, 4/8 and 4/7 in patients with lower baseline ALP in the HTD1801 1,000 mg BID, HTD1801 500 mg BID and placebo groups, respectively. As can be seen from Table 1, in both subgroups, 6 weeks of treatment with HTD1801 resulted in reduction of ALP, aspartate transaminase (AST) and alanine transaminase (ALT). In patients with elevated baseline ALP, a larger reduction in ALP was observed in contrast to subjects with lower baseline ALP. Similarly, greater reductions were also observed in AST and ALT in subjects with high baseline ALP. HTD1801 resulted in improvements in key measures of cholestasis and hepatocellular inflammation. Greater reductions were observed in patients with elevated ALP at baseline.

TABLE 1

Mean (SD) Change from Baseline to Week 6 in Key Measures of Cholestasis and Hepatocellular Inflammation

| | Baseline ALP ≥3x ULN | | | Baseline ALP <3x ULN | | |
|---|---|---|---|---|---|---|
| | HTD1801 1,000 mg BID (n=8) | HTD1801 500 mg BID (n=7) | Placebo (n=9) | HTD1801 1,000 mg BID (n=16) | HTD1801 500 mg BID (n=8) | Placebo (n=7) |
| Baseline ALP | 496 (169) | 509 (149) | 562 (226) | 254 (64) | 299 (52) | 224 (64) |
| Change from Baseline to Week 6 | | | | | | |
| ALP (U/L) | -185 (143) | -138 (134) | 86 (326) | -20 (106) | -13 (118) | 105 (145) |
| AST (U/L) | -37 (39) | -37 (49) | 17 (58) | -14 (26) | -18 (41) | 23 (39) |
| ALT (U/L) | -80 (56) | -48 (74) | 33 (79) | -30 (72) | -63 (104) | 50 (72) |

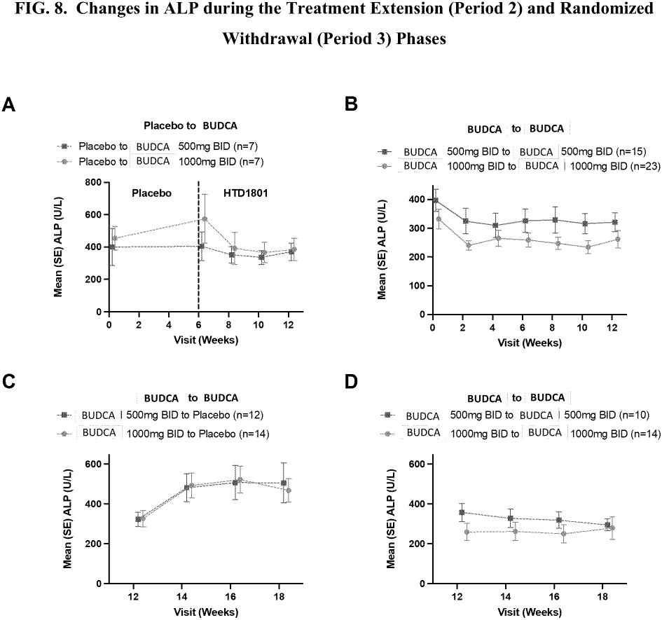

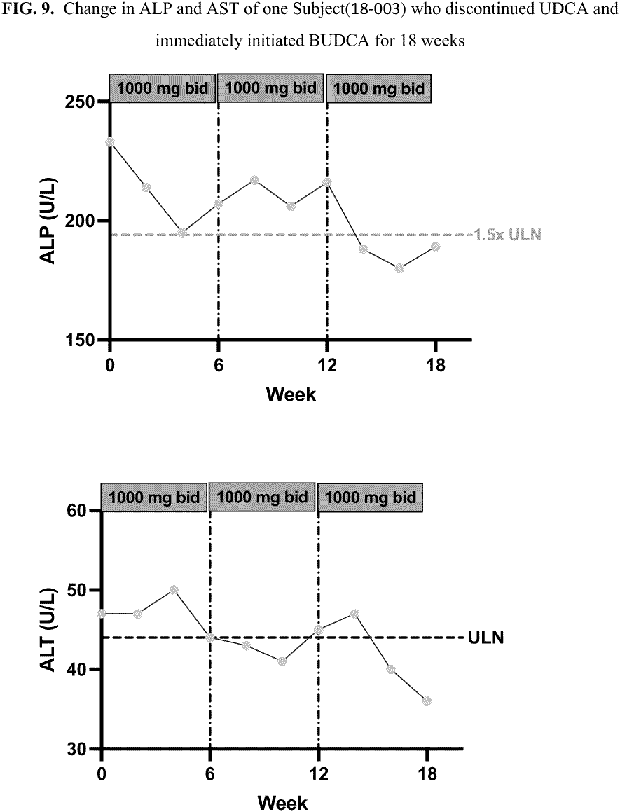

Changes in ALP during the Treatment Extension (Period 2) and Randomized Withdrawal (Period 3) Phases were also followed. Exemplary results are shown in FIG. 8.

In Period 2, patients initially randomized to placebo were re-randomized to receive either BUDCA 500 mg BID or BUDCA 1,000 mg BID, while those already on BUDCA continued their prior doses. As can be seen in FIG. 8A, re-randomized Placebo patients experienced decreases in ALP following initiation of BUDCA (mean [SD] change from Week 6 to Week 12, Placebo to BUDCA 500 mg BID: -34 [180.3] U/L, Placebo to BUDCA 1,000 mg BID: -189 [270.7] U/L). Patients who continued to receive BUDCA maintained their prior reductions in ALP, as shown in FIG. 8B, demonstrating ongoing durability of response with BUDCA. A dose response was evident for ALP, as their reductions in ALP from Week 6 to Week 12 upon transition from placebo were greater with BUDCA 1,000 mg BID treatment compared with 500 mg BID treatment.

Following the completion of Period 2, all patients were re-randomized to receive either Placebo or their prior dose of BUDCA. As can be seen in FIG. 8C, patients who had BUDCA withdrawn experienced a rise in ALP as early as Week 14 which was sustained to the end of Period 3. Patients who were maintained on BUDCA either continued to experience a further gradual decrease in ALP or sustained their ALP response achieved during Period 2, further indicative of a durable effect with BUDCA.

The ALP response associated with BUDCA treatment appeared to be reversible upon transition to placebo. In subjects who previously had been on BUDCA, there were substantial rises in ALP when they crossed over to placebo in Period 3 (rebound effect). Subjects receiving BUDCA 1,000 mg BID in Period 3 who had also received BUDCA 1,000 mg BID in Period 2 had the highest rate of response in meeting the secondary ALP endpoints during Period 3. Subjects receiving placebo in Period 3 had a very low response rate in meeting secondary ALP endpoints, regardless of prior treatment in Period 2; for example, the number of subjects achieving ALP <1.5 x ULN in the pooled active-to-active groups ranged between 4-6 depending on the week examined; the corresponding number in the pooled active-to-placebo groups was 0, 0, and 1 during Weeks 14, 16, and 18, respectively.

Similar trends were observed for GGT. Period 1 was followed by a dose-controlled extension period (Period 2). For subjects who previously had been on placebo in Period 1, there were substantial declines in GGT when they crossed over to active treatment in Period 2. Period 2 was followed by a randomized withdrawal period (Period 3). The efficacy associated with BUDCA treatment appeared reversible upon transition to placebo. In those subjects who had been on BUDCA in Periods 1 and 2, there were substantial rises in GGT when active treatment was withdrawn and they crossed over to placebo in Period 3 (rebound effect).

Durability of ALP and GGT effect was observed in those subjects who received active treatment for 12 consecutive weeks. Subjects receiving BUDCA 1,000 mg BID in Period 2 who had also received BUDCA 1,000 mg BID in Period 1 had a higher rate of response in meeting all secondary ALP endpoints compared to subjects receiving BUDCA 500 mg BID in both Periods 1 and 2. Subjects receiving BUDCA 1,000 mg BID in Period 3 who had also received BUDCA 1,000 mg BID in Period 2 (12 weeks cumulative treatment) had the highest rate of response in meeting the secondary ALP endpoints during Period 3. All these data suggest a dose response.

Mean total bilirubin, albumin, and platelets all remained stable and within normal range with all treatments throughout the study.

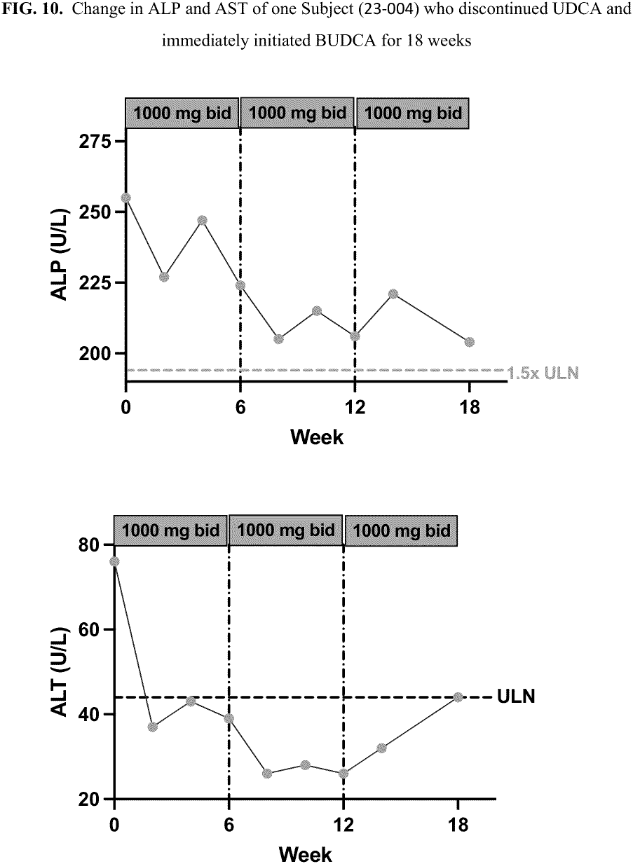

For subjects with prior UDCA use, ALP remained near Baseline values at Week 6 in subjects randomized to BUDCA treatment groups. In contrast, ALP increased substantially after six weeks in subjects randomized to placebo. 33% in the BUDCA 500 mg BID group with prior UDCA use achieved a 25% reduction in ALP compared to 0 subjects in placebo at week 6. For 1,000 mg BUDCA group with prior UDCA use, FIGS. 9 and 10 are comparative efficacy assessment results of prior UDCA versus BUDCA for 2 subjects who discontinued UDCA at Day -1 prior to Baseline (Day 0) and immediately initiated BUDCA 1,000 mg BID.

The 2 subjects (subjects 18-003, 23-004), UDCA dose prior to study baseline at 15 mg/kg or 12 mg/kg, with total duration of UDCA use (any dose) for 11 years and 2 years, respectively. Despite use of normal to high doses, ALP values at baseline remained high, 1.8 to 2.6-fold ULN suggesting a limited ALP response with their current UDCA dosage. Those two subjects received continuous treatment with our BUDCA for 18 weeks

Individual subject case profiles are described and summarized below.

Subject 18-003 received 18 weeks of continuous treatment with BUDCA 1,000 mg BID. This subject had been administering UDCA for ~ 11 years and was receiving a daily UDCA dose of 15 mg/kg which was discontinued at Day -1. On Day 0 (Baseline), the subject presented with an elevated ALP of 1.8-fold ULN and initiated BUDCA 1,000 mg BID which would yield a daily UDCA equivalent dose of 11 mg/kg — an approximate 25% lower dose of UDCA. Despite initiating a lower UDCA equivalent dose, ALP was decreased from 1.8-fold ULN at baseline to below 1.5-fold ULN after Week 12 through Week 18. GGT values fluctuated and were reduced by ~10% at Week 18. ALT (>ULN at baseline) and AST (within normal limits at baseline) similarly showed reductions with values within the normal range by Week 18. (FIG. 9).

Subject 23-004 received 18 weeks of continuous treatment with BUDCA 1,000 mg BID. This subject had been administering UDCA for ~ 2 years. Prior to baseline, Subject 23-004 was receiving a daily dose of 12 mg/kg UDCA which was discontinued at Day -1. On Day 0 (Baseline), the subject presented with a highly elevated ALP of 2.6-fold ULN and initiated treatment of BUDCA 1,000 mg BID, or a daily UDCA equivalent dose of 13 mg/kg. While receiving a similar UDCA equivalent dose, ALP was further reduced with BUDCA treatment. ALP was decreased from 2.6-fold ULN at baseline to below 1.6-fold ULN at Week 18. GGT showed a similar pattern with GGT values reduced by almost 50% and approaching the ULN at Week 18. ALT and AST (>ULN at baseline) and AST similarly showed reductions with values well within the normal range by Week 12. Apparent rises in both ALT and AST after Week 12 (Period 3) may be attributed to a SAE of intestinal obstruction reported at ~ Week 16 or Period 3. Treatment compliance was 80% at Week 18 due to the SAE (unable to take medication by mouth). (FIG. 10). This case indicates that subject on a similar UDCA equivalent dose with BUDCA showed marked reductions in markers of cholestasis.

In summary, both dose regimens of BUDCA were shown to be associated with significant reductions in serum ALP, an accepted marker of cholestasis, in subjects with PSC, as compared to placebo. Reductions in ALP were observed as early as Week 2 and were generally sustained, including in those who remained on BUDCA 1,000 mg BID through Week 18. In addition to ALP reductions, efficacy was supported by improvements in ALT, AST, and GGT following treatment with BUDCA compared with placebo. A dose response was evident, given that more subjects receiving BUDCA 1,000 mg BID met the secondary ALP endpoints than subjects receiving 500 mg BID. Furthermore, at Week 6, the percent change from Baseline in GGT was greater for BUDCA 1,000 mg BID (-40%) compared with the 500 mg BID dose (-21%). Further benefits of BUDCA on markers of liver injury and cholestasis can be achieved beyond that with UDCA alone. BUDCA has synergistic effect and can get better beneficial over UDCA on PSC patients with prior or no prior UDCA treatment.

BUDCA was safe and generally well tolerated, with no new safety signal identified, including few liver-related safety observations.

These data strongly support BUDCA as a safe and effective therapy for PSC.

Applicant’s disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant’s disclosure may be combined in any suitable manner in one or more embodiments. In the description, herein, numerous specific details are recited to provide a thorough understanding of embodiments of the invention. One skilled in the relevant art will recognize, however, that Applicant’s composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, manuscripts, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A method for treating primary sclerosing cholangitis (PSC), comprising administering to a subject in need thereof a therapeutically effective amount of a pharmaceutical composition comprising berberine ursodeoxycholate (BUDCA).

2. A method for reducing alkaline phosphatase (ALP) in a subject suffering from primary sclerosing cholangitis (PSC), comprising administering to a subject in need thereof a therapeutically effective amount of a pharmaceutical composition comprising berberine ursodeoxycholate (BUDCA).

3. A method for reducing cholestasis and/or hepatocellular inflammation in patients with primary sclerosing cholangitis (PSC) and elevated alkaline phosphatase (ALP), comprising administering to a subject in need thereof a therapeutically effective amount of a pharmaceutical composition comprising berberine ursodeoxycholate (BUDCA).

4. (canceled)

5. The method of claim 2, wherein the subject has not been previously treated for PSC with a therapeutic agent.

6. The method of claim 2, wherein the subject has not been previously treated with ursodeoxycholate (UDCA).

7. The method of claim 2, wherein the subject has been previously treated with ursodeoxycholate (UDCA).

8. The method of claim 2, wherein the subject suffers from an elevated ALP of above 3-fold ULN at baseline.

9. The method of claim 2, wherein the subject additionally suffers from hyperlipidemia and/or diabetes.

10. The method of claim 2, wherein the subject is administered from about 250 mg/day to about 5,000 mg/day of BUDCA.

11. The method of claim 10, wherein the subject is administered from about 500 mg/day to about 2,500 mg/day of BUDCA.

12. The method of claim 11, wherein the subject is administered from about 500 mg to about 1,500 mg BID of BUDCA.

13. The method of claim 11, wherein BUDCA is in the form of Form A.

14. (canceled)

15. The method of claim 11, wherein BUDCA is a hemi-nonahydrate.

16-25. (canceled)

26. The method of claim 3, wherein the subject has not been previously treated for PSC with a therapeutic agent.

27. The method of claim 3, wherein the subject has not been previously treated with ursodeoxycholate (UDCA).

28. The method of claim 3, wherein the subject has been previously treated with ursodeoxycholate (UDCA).

29. The method of claim 3, wherein the subject suffers from an elevated ALP of above 3-fold ULN at baseline.

30. The method of claim 3, wherein the subject additionally suffers from hyperlipidemia and/or diabetes.

31. The method of claim 3, wherein the subject is administered from about 250 mg/day to about 5,000 mg/day of BUDCA.

32. The method of claim 31, wherein BUDCA is in the form of Form A.
